(19) United States
(12) Patent Application Publication
(10) Pub. No.: US 2018/0268103 A1
(43) Pub. Date: Sep. 20, 2018

(54) SYSTEMS AND METHODS TO DETECT COPY NUMBER VARIATION

(71) Applicant: LIFE TECHNOLOGIES CORPORATION, Carlsbad, CA (US)

(72) Inventors: Fiona Hyland, San Mateo, CA (US); Rajesh Gottimukkala, Fremont, CA (US)

(21) Appl. No.: 15/866,578

(22) Filed: Jan. 10, 2018

Related U.S. Application Data
(62) Division of application No. 13/965,944, filed on Aug. 13, 2013, now abandoned.
Application No. 13/965,944 is a continuation of application No. 13/176,471, filed on Jul. 5, 2011, now abandoned.
(60) Provisional application No. 61/361,886, filed on Jul. 6, 2010.

(51) Int. Cl. G06F 19/22; C12Q 1/6809; G06F 19/18

(52) U.S. Cl. CPC G06F 19/22; C12Q 1/6809; G06F 19/18; C12Q 2537/165; C12Q 2535/122; C12Q 2537/16

(57) ABSTRACT

In one aspect, a system for implementing a copy number variation analysis method, is disclosed. The system can include a nucleic acid sequencer and a computing device in communications with the nucleic acid sequencer. The nucleic acid sequencer can be configured to interrogate a sample to produce a nucleic acid sequence data file containing a plurality of nucleic acid sequence reads. In various embodiments, the computing device can be a workstation, mainframe computer, personal computer, mobile device, etc. The computing device can comprise a sequencing mapping engine, a coverage normalization engine, a segmentation engine and a copy number variation identification engine.

### RELATED APPLICATIONS

This application claims priority pursuant to 35 U.S.C. § 119(e) to U.S. Provisional Patent Application Ser. No. 61/361,886, entitled “Method to Detect Copy Number Variation,” filed on Jul. 6, 2010, the entirety of which is incorporated herein by reference as if set forth in full.

### FIELD

The present disclosure generally relates to the field of nucleic acid sequencing including systems and methods for identifying genomic variants using nucleic acid sequencing data.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible for sequencing. These goals can be reached through the use of sequencing platforms and methods that provide sample preparation for larger quantities of samples of significant complexity, sequencing larger numbers of complex samples, and/or a high volume of information generation and analysis in a short period of time. Various methods, such as, for example, sequencing by synthesis, sequencing by hybridization, and sequencing by ligation are evolving to meet these challenges.

Research into fast and efficient nucleic acid (e.g., genome, exome, etc.) sequence assembly methods is vital to the sequencing industry as NGS technologies can provide ultra-high throughput nucleic acid sequencing. As such sequencing systems incorporating NGS technologies can produce a large number of short sequence reads in a relatively short amount time. Sequence assembly methods must be able to assemble and/or map a large number of reads quickly and efficiently (i.e., minimize use of computational resources). For example, the sequencing of a human size genome can result in tens or hundreds of millions of reads that need to be assembled before they can be further analyzed to determine their biological, diagnostic and/or therapeutic relevance.

Exemplary applications of NGS technologies include, but are not limited to: genomic variant (e.g., indels, copy number variations, single nucleotide polymorphisms, etc.) detection, resequencing, gene expression analysis and genomic profiling.

Of particular interest are copy number variations (CNVs), which have been observed in mammalian germline DNA and in tumor genomes. CNVs are being increasingly implicated as contributing factors in common disease states (for example, mental retardation and schizophrenia) and in cancer progression. In humans, more total nucleotides exhibit variation due to alterations in copy number than due to single nucleotide diversity. CNV detection has historically been done using comparative genomic hybridization, with one method measuring the log₂ ratio of test data intensity/control data intensity. Such methods have inherent limitations so there is a need for more flexible CNV detection and analysis approaches.

### SUMMARY

Systems, methods, software and computer-usable media for copy number variation determination from analyzing biomolecule-related sequence reads are disclosed. Biomolecule-related sequences can relate to proteins, peptides, nucleic acids, and the like, and can include structural and functional information such as secondary or tertiary structures, amino acid or nucleotide sequences, sequence motifs, binding properties, genetic mutations and variants, and the like.

Using nucleic acids as an example, in various embodiments, smaller nucleic acid sequence reads (e.g., NGS reads) can be assembled into larger sequences using an anchor-extension mapping method that initially maps (aligns) only a contiguous portion of each read to a reference sequence and then extends the mapping of the read at both ends of the mapped contiguous portion until the entire read is mapped (aligned). In various embodiments, a mapping score can be calculated for the read alignment using a scoring function, score (i, j)=M+mx, where M can be the number of matches in the extended alignment, x can be the number of mismatches in the alignment, and m can be a negative penalty for each mismatch. In various embodiments, the negative penalty, m, for each mismatch is user defined. In various embodiments, the negative penalty, m, for each mismatch is automatically determined by the algorithm/script/program implementing the anchor-extension mapping method to maximize the accuracy of the read alignment.

In various embodiments, the nucleic acid sequence read data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

In one aspect, a system for implementing a copy number variation analysis method, is disclosed. The system can include a nucleic acid sequencer and a computing device in communications with the nucleic acid sequencer. The nucleic acid sequencer can be configured to interrogate a sample to produce a nucleic acid sequence data file containing a plurality of nucleic acid sequence reads. In various embodiments, the computing device can be a workstation, mainframe computer, personal computer, mobile device, etc.

The computing device can be comprise a sequencing mapping engine, a coverage normalization engine, a segmentation engine and a copy number variation identification engine. The sequence mapping engine can be configured to align the plurality of nucleic acid sequence reads to a reference sequence, wherein the aligned nucleic acid sequence reads merge to form a plurality of chromosomal regions. The coverage normalization engine can be configured to divide each chromosomal region into one or more non-overlapping window regions, determine nucleic acid sequence read coverage for each window region and normalize the nucleic acid sequence read coverage determined for each window region to correct for bias.

The segmentation engine can be configured to convert the normalized nucleic acid sequence read coverage for each window region to discrete copy number states. The copy number variation identification engine can be configured to identify copy number variation in the chromosomal regions by utilizing the copy number states of each window region.

In one aspect, a computer-implemented method for identifying copy number variations, is disclosed. A nucleic acid sequence data file containing a plurality of nucleic acid sequence reads aligned to a reference sequence is received, wherein the aligned nucleic acid sequence reads together form a plurality of chromosomal regions. Each of the plurality of chromosomal regions are divided into one or more non-overlapping window regions. The nucleic acid sequence read coverage for each window region is determined. The nucleic acid sequence read coverage determined for each window region is normalized to correct for bias. The normalized nucleic acid sequence read coverage for each window region is converted to discrete copy number states. Copy number variation is identified in the chromosomal regions.

These and other features are provided herein.

### DRAWINGS

For a more complete understanding of the principles disclosed herein, and the advantages thereof, reference is now made to the following descriptions taken in conjunction with the accompanying drawings, in which:

FIG. 1 is a block diagram that illustrates a computer system, in accordance with various embodiments.

FIG. 2 is a schematic diagram of a system for reconstructing a nucleic acid sequence, in accordance with various embodiments.

FIG. 3 is a diagram showing a single sample CNV sequencing analysis pipeline, in accordance with various embodiments.

FIG. 4 is a schematic diagram of a system for CNV analysis, in accordance with various embodiments.

FIG. 5 is an exemplary flowchart showing a method for identifying CNV using a single sample approach, in accordance with various embodiments.

FIG. 6A is a depiction of a nucleic acid sequence that does not contain a copy number variant, in accordance with various embodiments.

FIG. 6B is a depiction of a nucleic acid sequence containing a copy number variant, in accordance with various embodiments.

FIG. 7 is an exemplary flowchart showing a method for identifying CNVs using a paired sample approach, in accordance with various embodiments.

FIG. 8A is an illustration of examples of genomic regions that show strong correlations between CNVs and changes of gene expression, in accordance with various embodiments.

FIG. 8B is an illustration of how large structural mutations are strongly correlated with tumor-specific changes in gene expression, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for copy number variation determination are described herein. According to the present teachings, nucleic acid sequencing technologies can be utilized for genome-wide interrogation of CNVs. In contrast to conventional approaches (e.g., array-based methods, etc.), with sequencing, genomic coverage data is available at single base resolution which allows for high levels of fidelity when researchers and clinicians search for genomic variants such as CNVs in a genome.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

It will be appreciated that there is an implied “about” prior to the temperatures, concentrations, times, number of bases, coverage, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, “a” or “an” means “at least one” or “one or more.”

A “system” denotes a set of components, real or abstract, comprising a whole where each component interacts with or is related to at least one other component within the whole.

A “biomolecule” is any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the SOLiD Sequencing System of Life Technologies Corp. provides massively parallel sequencing with enhanced accuracy. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference.

The phrase “sequencing run” refers to any step or portion of a sequencing experiment performed to determine some information relating to at least one biomolecule (e.g., nucleic acid molecule).

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

The phrase “ligation cycle” refers to a step in a sequence-by-ligation process where a probe sequence is ligated to a primer or another probe sequence.

The phrase “color call” refers to an observed dye color resulting from the detection of a probe sequence after a ligation cycle of a sequencing run.

The phrase “color space” refers to a nucleic acid sequence data schema where nucleic acid sequence information is represented by a set of colors (e.g., color calls, color signals, etc.) each carrying details about the identity and/or positional sequence of bases that comprise the nucleic acid sequence. For example, the nucleic acid sequence “ATCGA” can be represented in color space by various combinations of colors that are measured as the nucleic acid sequence is interrogated using optical detection-based (e.g., dye-based, etc.) sequencing techniques such as those employed by the SOLiD System. That is, in various embodiments, the SOLiD System can employ a schema that represents a nucleic acid fragment sequence as an initial base followed by a sequence of overlapping dimers (adjacent pairs of bases). The system can encode each dimer with one of four colors using a coding scheme that results in a sequence of color calls that represent a nucleotide sequence.

The phrase “base space” refers to a nucleic acid sequence data schema where nucleic acid sequence information is represented by the actual nucleotide base composition of the nucleic acid sequence. For example, the nucleic acid sequence “ATCGA” is represented in base space by the actual nucleotide base identities (e.g., A, T/or U, C, G) of the nucleic acid sequence.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The techniques of “paired-end,” “pairwise,” “paired tag,” or “mate pair” sequencing are generally known in the art of molecular biology (Siegel A. F. et al., Genomics. 2000, 68: 237-246; Roach J. C. et al., Genomics. 1995, 26: 345-353). These sequencing techniques can allow the determination of multiple “reads” of sequence, each from a different place on a single polynucleotide. Typically, the distance (i.e., insert region) between the two reads or other information regarding a relationship between the reads is known. In some situations, these sequencing techniques provide more information than does sequencing two stretches of nucleic acid sequences in a random fashion. With the use of appropriate software tools for the assembly of sequence information (e.g., Millikin S C. et al., Genome Res. 2003, 13: 81-90; Kent, W. J. et al., Genome Res. 2001, 11: 1541-8) it is possible to make use of the knowledge that the “paired-end,” “pairwise,” “paired tag” or “mate pair” sequences are not completely random, but are known to occur a known distance apart and/or to have some other relationship, and are therefore linked or paired in the genome. This information can aid in the assembly of whole nucleic acid sequences into a consensus sequence.

### Computer-Implemented System

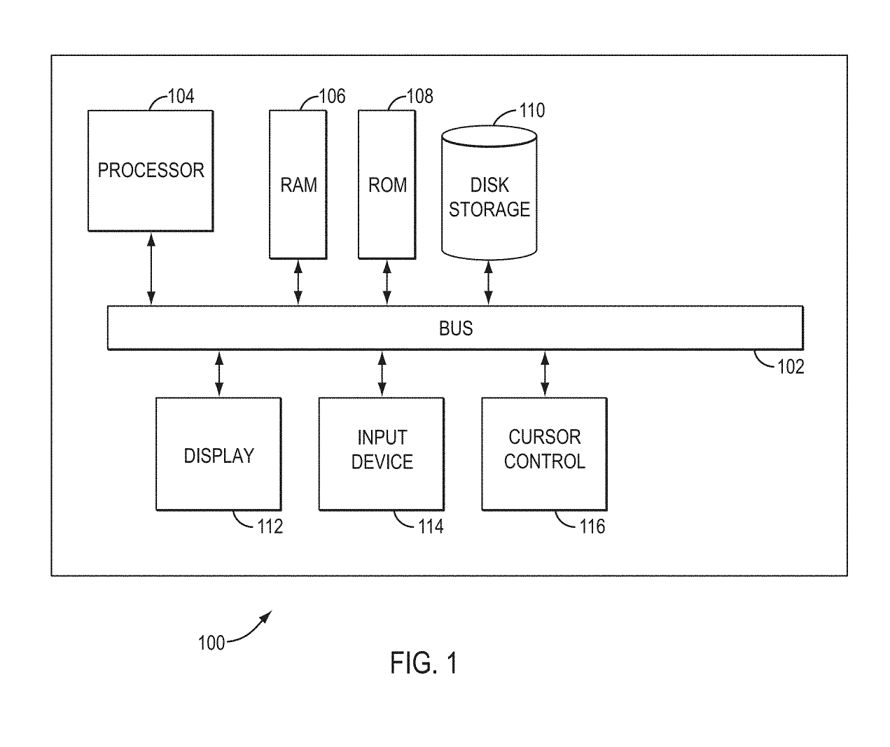

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to processor 104 for execution. For example, the instructions can initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 can carry the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

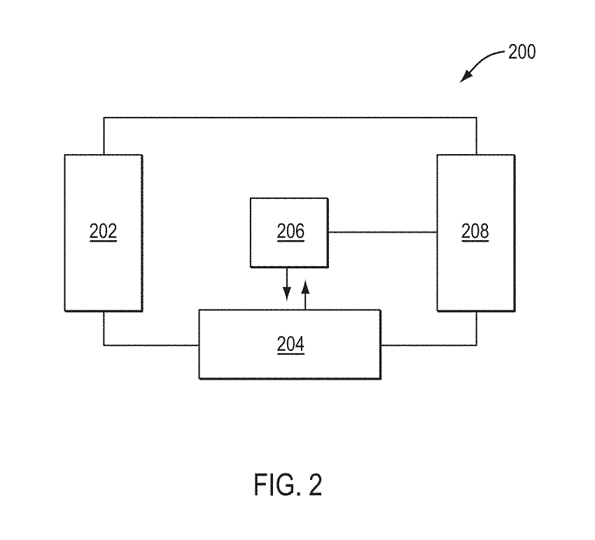

Various embodiments of nucleic acid sequencing platforms (i.e., nucleic acid sequencer) can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2007/066931 (application Ser. No. 11/737,308) and U.S. Patent Application Publication No. 2008/003571 (application Ser. No. 11/345,979) to McKernan, et al., which applications are incorporated herein by reference. Various embodiments of instrument 200 can provide for automated sequencing that can be used to gather sequence information from a plurality of sequences in parallel, i.e., substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion sensor, such as an ion sensitive layer overlying a CMOS, a current detector, or the like. The signal detection unit 206 can include an excitation system to cause a probe, such as a fluorescent dye, to emit a signal. The expectation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques. Ligation sequencing can include single ligation techniques, or change ligation techniques where multiple ligation are performed in sequence on a single primary nucleic acid sequence strand. Sequencing by synthesis can include the incorporation of dye labeled nucleotides, chain termination, ion/proton sequencing, pyrophosphate sequencing, or the like. Single molecule techniques can include continuous sequencing, where the identity of the nuclear type is determined during incorporation without the need to pause or delay the sequencing reaction, or staggered sequence, where the sequencing reactions is paused to determine the identity of the incorporated nucleotide.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

### CNV Detection Using Single Sample Approach

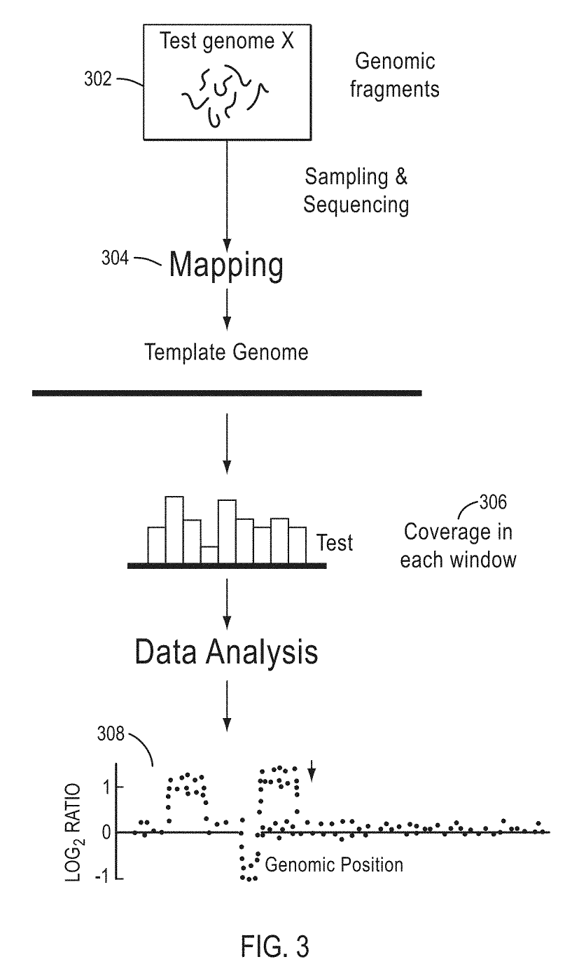

FIG. 3 is a diagram showing a single sample CNV sequencing analysis pipeline, in accordance with various embodiments. As shown herein, single-sample CNV analysis methods can be implemented as follows. In step 302, a single unique sample can be interrogated by a nucleic acid sequencing platform to generate a plurality of genomic (nucleic acid) fragment reads. The single sample represents the sample that is being analyzed for the presence or absence of CNVs and not a reference or control sample. In step 304, these genomic fragment reads are mapped to a reference genome (i.e., template genome) to form a plurality of chromosomal regions. In step 306, the chromosomal regions are divided into variable-sized genomic windows and read coverage is determined for each window. For coverage normalization, the variable-sized genomic windows are selected to contain a constant number of mappable positions (such an approach can smooth stochastic sampling noise). For an exemplary genome, the mappability for various run types (for example fragment or mate pair) and read lengths can be determined. This can be used to predict, for each genome position, whether it is likely to be capable of having reads uniquely map there or not based on the degree of homology or repetitiveness elsewhere in the genome. Within these windows, coverage can be further normalized based on predicted mappability and GC content of the window regions.

In various embodiments, a hidden markov model (HMM) can be used for segmentation, applying empirically derived filters to one or more contiguous window regions to call copy number states. In step 308, the copy number states of the window regions are determined and any copy number variations present can be detected for each genomic position (e.g., chromosomal region, etc.).

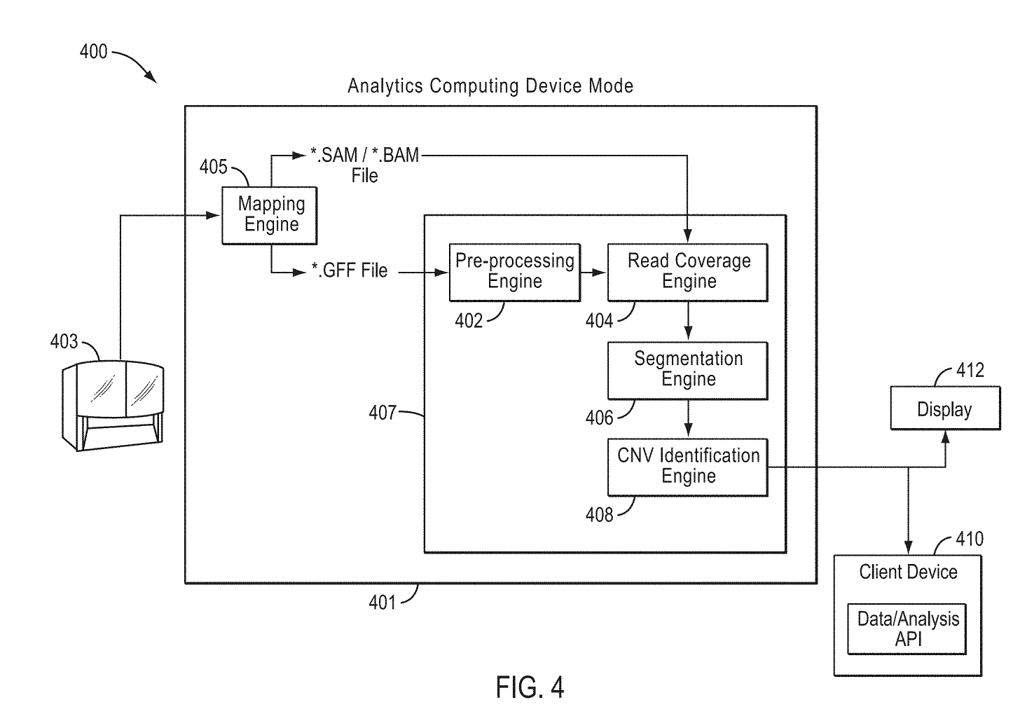

FIG. 4 is a schematic diagram of a system for CNV analysis, in accordance with various embodiments. As shown herein, system 400 can include an analytics computing device/node 401 in communications with a nucleic acid sequencer 403, a client device 410 (optional) and/or display terminal 412. The analytics computing device/node 401 can be configured to host a mapping engine 405 and a CNV detection program 407 comprised of a pre-processing engine 402, a read coverage engine 404, a segmentation engine 406 and a CNV identification engine 408. In various embodiments, the mapping engine 405 can be integrated as part of CNV detection program 407.

Nucleic acid sequencer 403 can be configured to sequence a plurality of nucleic acid fragments obtained from a single biological sample and generate a data file containing a plurality of fragment sequence reads that are representative of the genomic profile of the biological sample.

Client terminal 410 can be a thin client or thick client computing device. In various embodiments, client terminal 410 can have a web browser (e.g., INTERNET EXPLORER™ FIREFOX™, SAFARI™, etc) that can be used to control the operation of mapping engine 405, CNV detection program 407, pre-processing engine 402, read coverage engine 404, segmentation engine 406 and/or CNV identification engine 408 using a browser to control their function. For example, the client terminal 410 can be used to configure the operating parameters (e.g., mismatch constraint, quality value thresholds, window region sizing parameters, etc.) of the various engines, depending on the requirements of the particular application. Similarly, client terminal 410 can also display the results of the analysis performed by the mapping engine 405, CNV detection program 407, pre-processing engine 402, read coverage engine 404, segmentation engine 406 and/or CNV identification engine 408.

In various embodiments, the analytics computing device/node 401 can be a workstation, mainframe computer, personal computer, mobile device, etc.

Mapping engine 405 can be configured to receive nucleic acid (fragment) sequence read data output from nucleic acid sequencer 403, map the reads to a reference genome and output mapped reads data files (typically *.GFF, *.BAM or *.SAM data file formats) that contain a plurality of aligned nucleic acid sequence reads that together form a plurality of chromosomal regions. That is, in the nucleus of each cell, the DNA molecule is packaged into thread-like structures called chromosomes. Each chromosome is made up of DNA tightly coiled many times around proteins called histones that support its structure. Each chromosome has a constriction point called the centromere, which divides the chromosome into two sections, or “arms.” The short arm of the chromosome is labeled the “p arm.” The long arm of the chromosome is labeled the “q arm.” In various embodiments, the p-arm and q-arm are treated as two different chromosomal regions to avoid the region around the centromere which can contain repetitions and lead to false positive CNV calls.

The pre-processing engine 402 can be configured to receive nucleic acid sequence read data files that do not contain read coverage information (e.g., *.GFF files, etc.) from mapping engine 405, determine the read coverage for each base position of the plurality of chromosomal regions formed by the aligned reads and output a sequence read data file containing chromosomal regions and their associated read coverage information.

The read coverage engine 404 can be configured to receive a nucleic acid sequence read data file (e.g., *.BAM/*.SAM files, pre-processed *.GFF files, etc.) containing a plurality of reference sequence aligned nucleic acid sequence reads that together form a plurality of chromosomal regions (with associated read coverage information), divide each of the chromosomal regions into one or more non-overlapping variable size window regions, determine sequence read coverage for each of the variable sized window regions and normalize the sequence read coverage determined for each window region to correct for bias (such as GC bias).

In various embodiments, each of the window regions are sized so that they contain about the same number of uniquely mappable bases. That is, the mappability of each of the bases that comprise the window regions are determined by generating mappability files (which are essentially a representation of reads from the reference that are mapped back to the reference) for each window region. The mappability files have one row per every position, indicating whether each position is or is not uniquely mappable. In various embodiments, a position is classified as uniquely mappable if the coverage at that position is greater than a threshold value (typically threshold value=0). The positions with coverage less than or equal to the threshold are classified as non-uniquely mappable. Coverage per position is calculated using the alignments of reads simulated from the reference mapped back to the same reference.

In various embodiments, the read coverage for each window region can be represented as a local Sᵢ score which is calculated using Equation 1:

Sᵢ=log₂(Observedᵢ/Expectedᵢ) Equation 1:
where:
   Observedᵢ=Actual read coverage observed for that window region
   Expectedᵢ=Average read coverage for all the window regions of the sequenced sample

GC content is the number of G or C bases compared to the total number of bases in a particular region. GC bias occurs in regions of the genome where the percentage of GC content is either high or low which can cause the observed read coverage observed for that region to be artificially low (GC biased).

In various embodiments, a GC bias correction algorithm can be applied (by the read coverage engine 404) to normalize the effect of GC content by scaling the coverage of the window regions with very high GC content to match that of the median coverage. The scaling factors for the window regions can be computed for every chromosome arm during runtime by the algorithm.

In various embodiments, the GC bias correction algorithm is as follows:

Read coverage is calculated (average coverage of all the bases in the window) for every window region in a chromosome.

GC_{fraction} is calculated by dividing the total number of G or C bases in a given window region by the total number of bases in that window region.

All the window regions are binned according to the GC_{fraction} (i.e., all the window regions with GC_{fraction} from 0 to 0.05 is put in bin 1, 0.05 to 0.1 in bin 2, 0.1 to 0.15 in bin 3, etc.). Read coverage of each bin is then calculated as the median read coverage of all the window regions in the bin.

To normalize for GC bias, a GC bias scaling factor can be applied to each window region where:
GC bias scaling factor=Bₘₐₓ/read coverage of the bin that each window region belongs to
(where Bₘₐₓ is the read coverage of the bin with the highest read coverage)

The GC bias scaling factor can be applied to all the window regions in every bin. Read coverage of a window region is thus scaled (normalized)=original coverage of window region*GC bias scaling factor of that window region.

The segmentation engine 406 can be configured to convert the normalized nucleic acid sequence read coverage for each window region to discrete copy number states using a stochastic modeling algorithm. In various embodiments, a Hidden Markov Modeling (HMM) algorithm is applied to convert the normalized read coverage for each window region to discrete copy number states.

The CNV identification engine 408 can be configured to identify putative CNVs in the chromosomal regions by utilizing the copy number states of each window region. For example, as shown in step 308 of FIG. 3.

In various embodiments, all adjacent windows with the same copy number can be merged into a segment for CNV reporting purposes. In various embodiments, CNV identification engine 408 can be further configured to filter the window regions before they are merged into a segment to meet minimum segment length requirements or window region mappability thresholds.

It should be understood, however, that the various modules/engines shown as being part of the system 400 can be combined or collapsed into a single module/engine, depending on the requirements of the particular application or system architecture. Moreover, in various embodiments, the system 200 can comprise additional modules, engines or components as needed by the particular application or system architecture.

In various embodiments, the system 400 can be configured to process the nucleic acid reads in color space. In various embodiments, system 400 can be configured to process the nucleic acid reads in base space. It should be understood, however, that the system 400 disclosed herein can process or analyze nucleic acid sequence data in any schema or format as long as the schema or format can convey the base identity and position of the nucleic acid sequence.

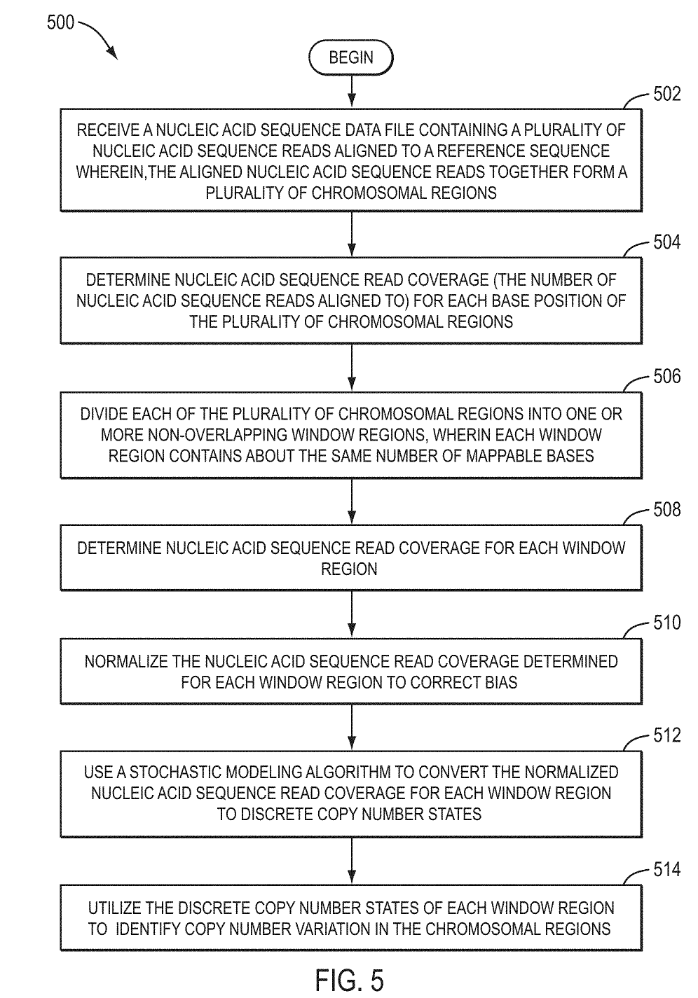

FIG. 5 is an exemplary flowchart showing a method for identifying CNV using a single sample approach, in accordance with various embodiments.

In step 502, a nucleic acid sequence data file containing a plurality of nucleic acid sequence reads aligned to a reference sequence is received. In various embodiments, the aligned nucleic acid sequence reads together form a plurality of chromosomal regions.

As discussed above, in the nucleus of each cell, the DNA molecule is packaged into thread-like structures called chromosomes. Each chromosome is made up of DNA tightly coiled many times around proteins called histones that support its structure. Each chromosome has a constriction point called the centromere, which divides the chromosome into two sections, or “arms.” The short arm of the chromosome is labeled the “p arm.” The long arm of the chromosome is labeled the “q arm.” In various embodiments, the p-arm and q-arm are treated as two different chromosomal regions to avoid the region around the centromere which can contain repetitions and lead to false positive CNV calls.

In step 504, the nucleic acid sequence read coverage (the number of nucleic acid sequence reads aligned to each base) for each base position of the plurality of chromosomal region can be optionally determined. This pre-processing step is typically performed on nucleic acid sequence read data files that do not contain read coverage information (e.g., *.GFF files, etc.).

In step 506, each of the plurality of chromosomal regions is divided into one or more non-overlapping window regions, wherein each window region contains about the same number of mappable bases. As discussed above, the mappability of the each of the bases that comprise the window regions are determined by generating mappability files (which are essentially a representation of reads from the reference that are mapped back to the reference) for each window region. The mappability files have one row per every position, indicating whether each position is or is not uniquely mappable. In various embodiments, a position is classified as uniquely mappable if the coverage at that position is greater than a threshold value (typically threshold value=0). The positions with coverage less than or equal to the threshold are classified as non-uniquely mappable. Coverage per position is calculated using the alignments of reads simulated from the reference mapped back to the same reference.

In step 508, the nucleic acid sequence read coverage for each window region is determined. In various embodiments, the read coverage for each window region can be represented as a local Sᵢ score which is calculated using Equation 1, as shown above.

In step 510, the nucleic acid sequence read coverage determined for each window region is normalized to correct for bias (such as GC bias). In various embodiments, the read coverage for each window region can be normalized for GC bias through the application of a GC bias scaling factor to each window region where:
GC bias scaling factor=Bₘₐₓ/read coverage of the bin that each window region belongs to
(where Bₘₐₓ is the read coverage of the bin with the highest read coverage)

The GC bias scaling factor can be applied to all the window regions in every bin. Read coverage of a window region is thus scaled (normalized)=original coverage of window region*GC bias scaling factor of that window region.

In step 512, a stochastic modeling algorithm is utilized to convert the normalized nucleic acid sequence read coverage for each window region to discrete copy number states. In various embodiments, a Hidden Markov Modeling (HMM) algorithm is applied to convert the normalized read coverage for each window region to discrete copy number states.

In step 514, the discrete copy number states of each window region can be utilized to identify copy number variation in the chromosomal regions. In various embodiments, all adjacent window regions with the same copy number can be merged into a segment for CNV reporting purposes. In various embodiments, window regions can be filtered before they are merged into a segment to meet minimum segment length requirements or window region mappability thresholds, etc.

In various embodiments, the methods of the present teachings may be implemented in a software program and applications written in conventional programming languages such as C, C++, etc. The coded method may implement an automated or partially-automated approach for detecting CNVs in selected sample sequence data obtained for example using a sequencing system. In various embodiments, such an approach can utilize empirically derived normalization for CG content as well as a Hidden Markov Model (HMM) for segmentation with a series of empirically derived filters for assigning segments to copy number variants.

### CNV Detection Using Paired Sample Approach

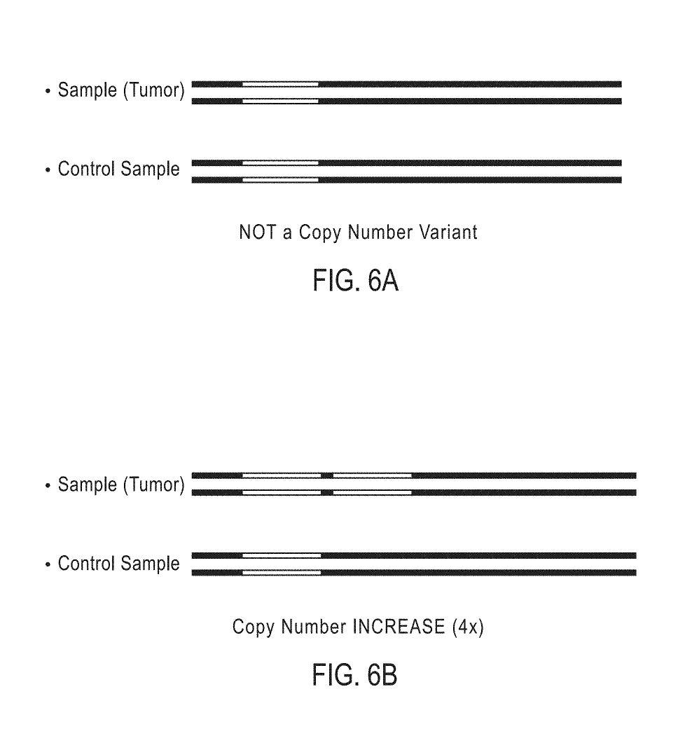

Paired sample CNV detection shares many of the sample steps/operations as those used in single sample CNV detection. However, as depicted in FIGS. 6A and 6B, in the case of paired-sample CNV detection, rather than comparing to the predicted mappabilty of the genome, the coverage of the test sample can be normalized by comparing it to the coverage of a control sample. Using such an approach desirably addresses systematic issues such as mappability, GC content, which may be expected to be similar between both samples, thus simplifying normalization.

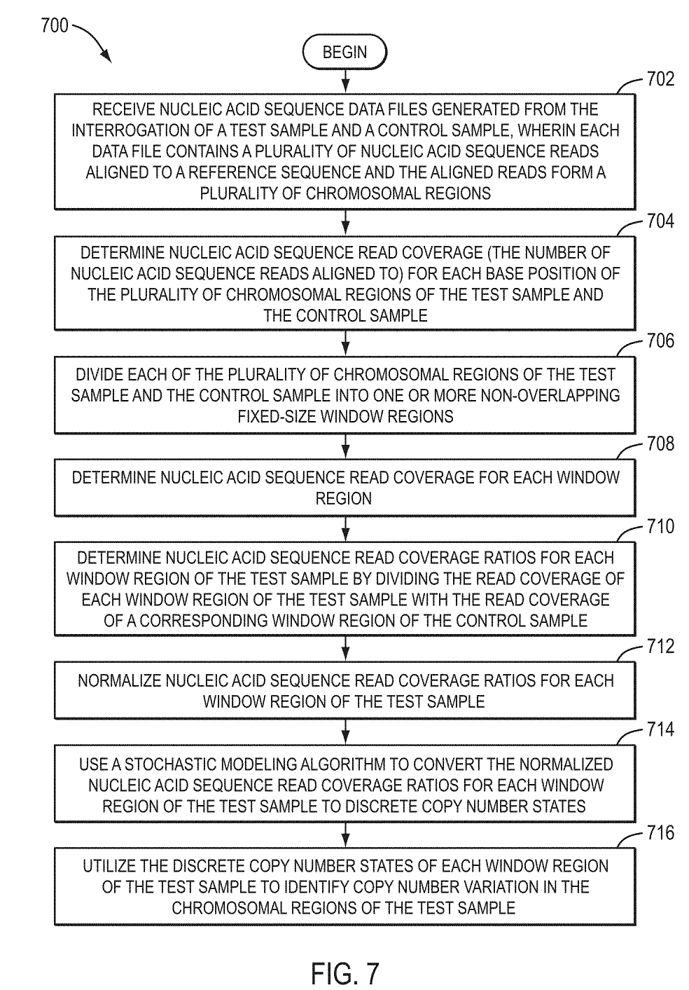

FIG. 7 is an exemplary flowchart showing a method for identifying CNVs using a paired sample approach, in accordance with various embodiments.

In step 702, nucleic acid sequence data files generated from the interrogation of a test sample and a control sample is received. Each data file contains a plurality of nucleic acid sequence reads aligned to a reference sequence and the aligned reads form a plurality of chromosomal regions. In various embodiments, the test sample and control sample nucleic acid sequenced reads can be stored in a single nucleic acid sequence data file.

In step 704, nucleic acid sequence read coverage can be determined for each base position of the plurality of chromosomal regions of the test sample and the control sample.

In step 706, each of the plurality of chromosomal regions of the test sample and the control sample can be divided into one or more non-overlapping fixed-size window regions. In various embodiments, the window size can be variable and determined for example by fixing the number of positions of a control sample with coverage.

In step 708, nucleic acid sequence read coverage for each window region can be determined. To adjust for coverage differences in the samples, coverage of each window can be normalized by the mean coverage of that sample. Using such an approach, it may be desirable to sequence both samples (test and control) under the same conditions (e.g. both mate pair, both the same tag length). In various embodiments, the read coverage for each window region can be represented as a local Sᵢ score which is calculated using Equation 1, as shown above.

In step 710, nucleic acid sequence read coverage ratios for each window region of the test sample can determined by dividing the read coverage of each window region of the test sample with the read coverage of a corresponding window region of the control sample. For example, in accordance with various embodiments, the read coverage for a window region from a particular position on Chromosome 7 for the test sample can be divided with the read coverage for a window region from a similar or identical position on Chromosome 7 from the control sample to arrive at a read coverage ratio for the test sample window region.

In step 712, nucleic acid sequence read coverage ratios can be determined for each window region of the test sample.

In step 714, a stochastic modeling algorithm can be used to convert the normalized nucleic acid sequence read coverage ratios for each window region of the test sample to discrete copy number states. In various embodiments, a Hidden Markov Modeling (HMM) algorithm is applied to convert the normalized read coverage ratios for each window region to discrete copy number states.

In step 716, the discrete copy number states of each window region of the test sample can be utilized to identify copy number variation in the chromosomal regions of the test sample. In various embodiments, all adjacent window regions with the same copy number can be merged into a segment for CNV reporting purposes. In various embodiments, window regions can be filtered before they are merged into a segment to meet minimum segment length requirements or window region mappability thresholds, etc.

In various embodiments, the methods of the present teachings may be implemented in a software program and applications written in conventional programming languages such as C, C++, etc. The coded method may implement an automated or partially-automated approach for detecting CNVs in selected sample sequence data obtained for example using a sequencing system. In various embodiments, such an approach can utilize empirically derived normalization for CG content as well as a Hidden Markov Model (HMM) for segmentation with a series of empirically derived filters for assigning segments to copy number variants.

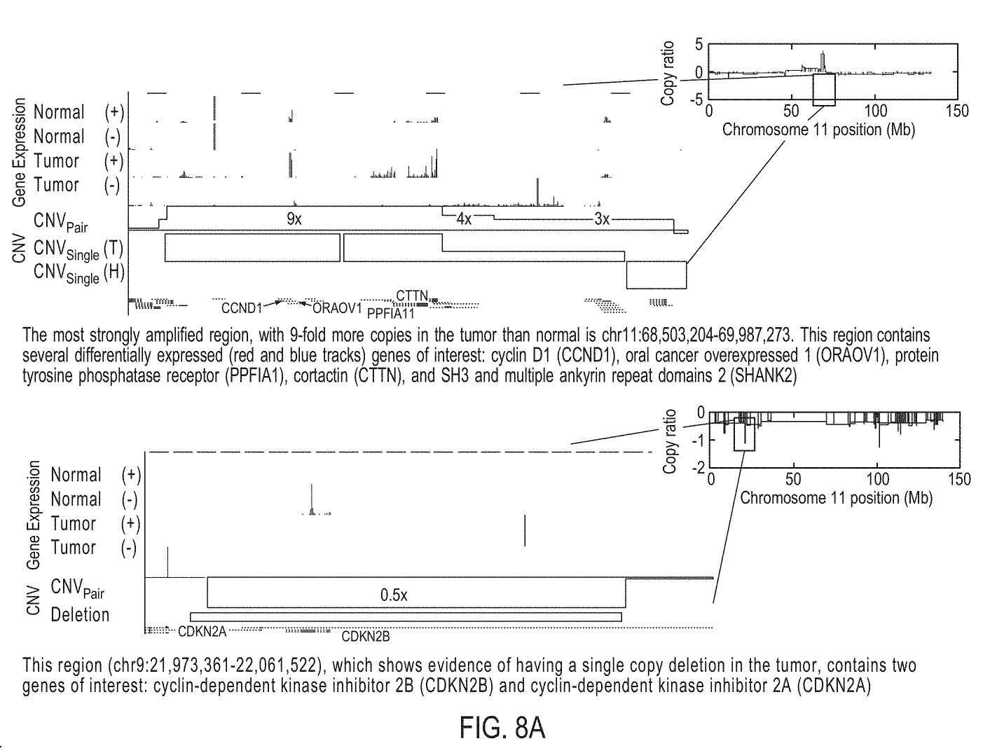

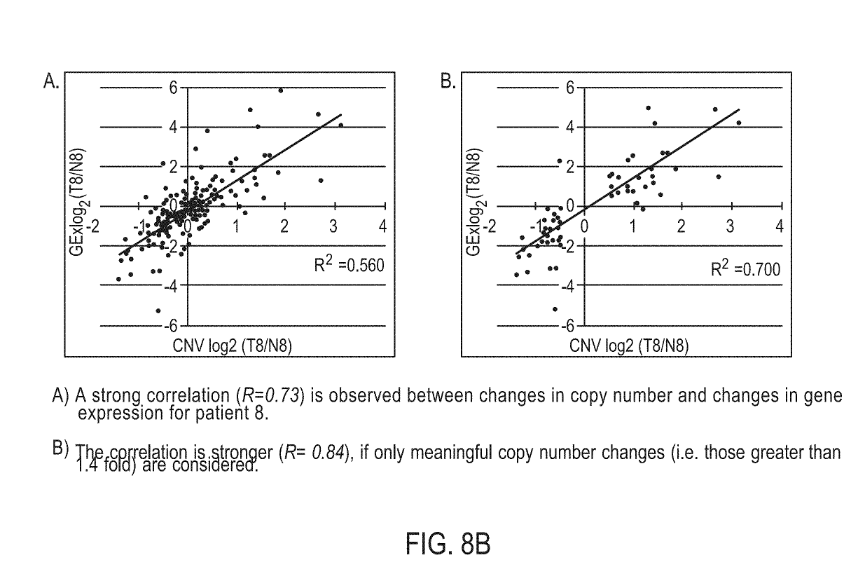

As shown in FIGS. 8A and 8B, no CNVs are observed in an exemplary data set with a control data set that uses the same sample to normalize itself to. This suggests that false positives do not present an issue where 3 consecutive window regions are used to call a CNV, and 4 CNVs were observed when 2 consecutive window regions were used, suggesting a very low false positives rate for paired-sample CNV detection.

In the exemplary comparison, Tumor and Normal data oral squamous cell carcinoma (OSCC) samples can be sequenced and a matched normal sample (shown with an exemplary 0.8× coverage using the SOLiD Sequencing System). In various embodiments, whole transcriptome analysis of the tumor and normal samples may also be conducted using RNA-based protocols and examining the correlation between copy number variation and changes in gene expression.

In various embodiments, a significantly positive correlation between CNV and gene expression can be observed. Such results suggest that gene duplication and deletion are key mechanisms driving the transcriptional profile changes of these tumor samples. Thus, from the identified CNV segments the method and operations of the present teachings may offer insight into genes associated with the initiation or progression of cancer.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

What is claimed is:

1. A system for copy number variation analysis, comprising:
a nucleic acid sequencer configured to interrogate a sample to produce a nucleic acid sequence data file containing a plurality of nucleic acid sequence reads;
a computing device in communications with the nucleic acid sequencer, comprising;
a sequence mapping engine configured to align the plurality of nucleic acid sequence reads to a reference sequence, wherein the aligned nucleic acid sequence reads merge to form a plurality of chromosomal regions;
a coverage normalization engine configured to,
divide each chromosomal region into one or more non-overlapping window regions,
determine nucleic acid sequence read coverage for each window region, and
normalize the nucleic acid sequence read coverage determined for each window region to correct for bias;
a segmentation engine configured to convert the normalized nucleic acid sequence read coverage for each window region to discrete copy number states;
a copy number variation identification engine configured to identify copy number variation in the chromosomal regions by utilizing the copy number states of each window region.

2. The system for copy number variation analysis, as recited in claim 1, wherein each window region contains about the same number of mappable bases.

3. The system for copy number variation analysis, as recited in claim 1, wherein each window region contains about 5000 mappable bases.

4. The system for copy number variation analysis, as recited in claim 1, further including a pre-processing engine configured to determine the nucleic acid sequence read coverage for each base position of the plurality of chromosomal regions.

5. The system for copy number variation analysis, as recited in claim 1, wherein the segmentation engine utilizes a stochastic modeling algorithm to convert the nucleic acid sequencing read coverage of each window region to discrete copy number states.

6. The system for copy number variation analysis, as recited in claim 5, wherein the stochastic modeling algorithm is a Hidden Markov Model algorithm.

7. The system for copy number variation analysis, as recited in claim 1, wherein the segmentation engine is configured to merge adjacent window regions with the same copy number states together.

8. The system for copy number variation analysis, as recited in claim 1, wherein the copy number variation identification engine is configured to designate window regions with copy number states of greater than two as copy number amplifications.

9. The system for copy number variation analysis, as recited in claim 8, wherein the copy number variation identification engine is configured to designate window regions with copy number states of less than two as copy number deletions.

10. A system for copy number variation analysis, comprising:
a nucleic acid sequencer that interrogates a sample and produces a plurality sequences reads from the sample; and
a computing device in communication with the sequencer and configured to:
obtain the sequence reads from the sequencer,
perform alignments of the sequence reads against a reference sequence,
divide the reference aligned sequence reads into a plurality of window regions and determining read coverage for each window region,
determine putative copy number variations in the window regions by applying a stochastic modeling algorithm to convert the read coverage of each window region into copy number states, and
output copy number variations in the reference mapped sequence reads.

11. A computer-implemented method for identifying copy number variations, comprising:
receiving a nucleic acid sequence data file containing a plurality of nucleic acid sequence reads aligned to a reference sequence, wherein the aligned nucleic acid sequence reads together form a plurality of chromosomal regions;
dividing each of the plurality of chromosomal regions into one or more non-overlapping window regions;
determining nucleic acid sequence read coverage for each window region;
normalizing the nucleic acid sequence read coverage determined for each window region to correct for bias;
converting the normalized nucleic acid sequence read coverage for each window region to discrete copy number states; and
identifying copy number variation in the chromosomal regions.

12. The computer-implemented method for identifying copy number variations, as recited in claim 11, further including:
determining nucleic acid sequence read coverage for each base position of the plurality of chromosomal regions.

13. The computer-implemented method for identifying copy number variations, as recited in claim 11, wherein a stochastic modeling algorithm is utilized to convert the nucleic acid sequencing read coverage of each window region to discrete copy number states.

14. The computer-implemented method for identifying copy number variations, as recited in claim 13, wherein the stochastic modeling algorithm is a Hidden Markov Model algorithm.

15. The computer-implemented method for identifying copy number variations, as recited in claim 11, wherein each window region includes about the same number of mappable bases.

16. The computer-implemented method for identifying copy number variations, as recited in claim 11, wherein each window region includes about 5000 mappable bases.

17. The computer-implemented method for identifying copy number variations, as recited in claim 11, further including:
merging adjacent window regions with the same copy number states together.

18. The computer-implemented method for identifying copy number variations, as recited in claim 11, further including:
designating window regions with copy number states of greater than two as copy number amplifications.

19. The computer-implemented method for identifying copy number variations, as recited in claim 11, further including:
designating window regions with copy number states of less than two as copy number deletions.

20. (canceled)
